SEQUENCE LISTING (6 sequences)

SEQ ID NO: 1 - DNA, 1223 bp, Artificial Sequence
  Synthetic Construct
gccaggaccctggaaggaagcaggatggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggtgaggggtttgataaagtcagggaagcagaagatagcccccaacacatgtgactggggggatggtcaacaagaaaggaatggaaggccccagaaaaccaggaggaagaggaggagcgtgcagaactgaatcagtcggaggaacctgaggcaggcgagagtagtactggagggccttgaggggcccacagacagatcccatccatcag

SEQ ID NO: 2 - DNA, 1194 bp, Artificial Sequence
  Synthetic Construct
aggaagcaggatggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggcctcaggaccagggaacctacagctgtgtggccacccattccagccacgggccccaggaaagccgtgctgtcagcatcagcatcatcgaaccaggcgaggaggggccaactgcaggctctgtgggaggatcagggctgggaactctagccctggccctggggatcctgggaggcctggggacagccgccctgctcattggggtcatcttgtggcaaaggcggcaacgccgaggagaggagaggaaggccccagaaaaccaggaggaagaggaggagcgtgcagaactgaatcagtcggaggaacctgaggcaggcgagagtagtactggagggccttgaggggcccacagacagatccca

SEQ ID NO: 3 - DNA, 1201 bp, Artificial Sequence
  Synthetic Construct
aggaagcaggatggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggtgaggggtttgataaagtcagggaagcagaagatagcccccaacacatgtgactggggggatggtcaacaagaaaggaatggaaggccccagaaaaccaggaggaagaggaggagcgtgcagaactgaatcagtcggaggaacctgaggcaggcgagagtagtactggagggccttgaggggcccacagacagatccca

SEQ ID NO: 4 - DNA, 1149 bp, Artificial Sequence
  Synthetic Construct
aggaagcaggatggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggcctcaggaccagggaacctacagctgtgtggccacccattccagccacgggccccaggaaagccgtgctgtcagcatcagcatcatcgaaccaggcgaggaggggccaactgcaggtgaggggtttgataaagtcagggaagcagaagatagcccccaacacatgtgactggggggatggtcaacaagaaaggaatggaaggccccagaaaaccaggaggaagaggaggagcgtgcagaactgaatcagtcggaggaacctgaggcaggcgagagtagtactggagggccttgaggggcccacagacagatccca

SEQ ID NO: 5 - DNA, 1249 bp, Artificial Sequence
  Synthetic Construct
aggaagcaggatggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtagtggaagaaagcaggagaagtagaaaacggccctgtgaacaggaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggtgaggggtttgataaagtcagggaagcagaagatagcccccaacacatgtgactggggggatggtcaacaagaaaggaatggaaggccccagaaaaccaggaggaagaggaggagcgtgcagaactgaatcagtcggaggaacctgaggcaggcgagagtagtactggagggccttgaggggcccacagacagatccca

SEQ ID NO: 6 - DNA, 1164 bp, Artificial Sequence
  Synthetic Construct
atggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcggagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggggtgcccttgccccttccccccagccctgtgctgatcctccctgagatagggcctcaggaccagggaacctacagctgtgtggccacccattccagccacgggccccaggaaagccgtgctgtcagcatcagcatcatcggtgagacctctccccaagccctacagaccctgggactagggtgcaggacagcacaggctctaatttcctgccccattctggccttatccctaacagccaccccacctctccctccatgcacccacacccaagcctcccctgccccacccaaattctgccaagagagcagccaagcctctcccttcttccctctgagctaa

(19) United States
(12) Patent Application Publication
(10) Pub. No.: US 2019/0202886 A1
(43) Pub. Date: Jul. 4, 2019

(54) ONCOLYTIC VIRUSES COMPRISING esRAGE AND METHODS OF TREATING CANCER

(71) Applicant: Ohio State Innovation Foundation, Columbus, OH (US)

(72) Inventors: Balveen KAUR, Houston, TX (US); Ji Young YOO, Houston, TX (US)

(21) Appl. No.: 16/300,452

(22) PCT Filed: May 11, 2017
(86) PCT No.: PCT/US17/32300

§ 371 Date: Nov. 9, 2018

Related U.S. Application Data
(60) Provisional application No. 62/334,589, filed on May 11, 2016.

(51) Int. Cl. C07K 14/705; C12N 7/00; A61P 35/00; A61K 35/763; A61K 35/768

(52) U.S. Cl. CPC C07K 14/705; C12N 7/00; A61P 35/00; A61K 38/00; A61K 35/768; C12N 2710/16132; C12N 2710/16143; A61K 35/763

(57) ABSTRACT

Disclosed are novel modified or engineered oncolytic viruses comprising an esRAGE gene and methods for using said oncolytic virus for the treatment of a cancer.

### I. BACKGROUND

The ability of oncolytic viruses to specifically target cancer cells either through viral lysis of the cell or recruitment of host immune responses has moved treatment of cancers with administration of an oncolytic virus to the forefront of cutting edge cancer therapies. However, the efficacy of oncolytic viral therapy can be inhibited by early innate immune responses to viral infection reduce oHSV replication, tumor destruction, and efficacy. Moreover, inflammatory signals can upregulate expression of the receptor for advanced glycation endproducts (RAGE) on endothelial cells. Binding of any of the RAGE ligands to RAGE causes proliferation, migration, invasion, angiogenesis of endothelial cells. Accordingly, what are needed are new therapies and methods of treatment that reduce, inhibit or prevent RAGE signaling and lead to the escape and proliferation of cancer cells in a subject receiving oncolytic viral therapy.

### II. SUMMARY

Disclosed are methods and compositions related to oncolytic viruses expressing endogenous secretory receptor for advanced glycation endproducts (esRAGE).

In one aspect disclosed herein are modified oncolytic viruses; wherein the oncolytic virus been modified to encode and express the endogenous secretory receptor for advanced glycation endproducts (esRAGE) gene or a functional fragment or variant thereof comprising at least 90% sequence identity to SEQ ID NO: 1, SEQ ID NO: 2, SEQ ID. NO: 3, SEQ ID NO: 4, SEQ ID NO: 5, and/or SEQ ID NO: 6.

In one aspect, the modified oncolytic viruses of any preceding aspect can comprise a viral backbone derived from a modified or engineered Adenovirus, Adeno-associated virus, Herpes Simplex virus-1, Herpes Simplex virus-2, Varicella-Zoster virus, Epstein-Barr virus, Cytomegalovirus, Human Herpes virus-6, Variola virus, Vaccinia virus, Molluscum contagiosum virus, Orf virus, Reovirus, Rotavirus, Enterovirus, Senecavirus, Poliovirus, Coxsackie virus, Rhinovirus, Hepatitis A virus, foot-and-mouth disease virus, Togavirus, Alphavirus, Semliki Forest virus, Eastern Equine Encephalitis virus, Sindbis virus, Rubella virus, Coronavirus, Flavivirus Hepatitis C virus, Japanese Encephalitis virus, St. Louis Encephalitis virus, Murray Valley fever virus, Yellow Fever virus, West Nile virus, Zika virus, Dengue virus, Ebola virus, Marburg virus, Arenavirus, Lassa fever virus, Lymphocytic choriomeningitis virus, Pichinde virus, Junin virus, Machupo virus, Hantaan virus, Rift Valley fever virus, Paramyxovirus, human parainfluenza virus, mumps virus, simian virus 5, measles virus, vesicular stomatitis virus, rabies virus, Respiratory syncytial virus, Orthomyxovirus, Influenza virus A, Influenza virus B, Influenza C virus, Hepatitis D virus, Simian Immunodeficiency virus, Human Immunodeficiency virus type-1, and Human Immunodeficiency virus type-2, Rous sarcoma virus, Human T-cell Leukemia virus type-1 Simian foamy virus, Hepatitis B virus, Hepatitis E virus, Human Papilomavirus, or Polyomavirus.

Also disclosed are pharmaceutical composition comprising the oncolytic virus of any preceding aspect and a pharmaceutical carrier.

Also disclosed are methods of treating a subject with cancer comprising administering to the subject the oncolytic virus of any preceding aspect.

In one aspect, disclosed herein are methods of treating a subject with a cancer comprising administering to the subject modified oncolytic virus; wherein the oncolytic virus been modified to encode and express the endogenous secretory receptor for advanced glycation endproducts (esRAGE) gene or a functional fragment thereof comprising at least 90% sequence identity to SEQ ID NO: 1, SEQ ID NO: 2, SEQ ID. NO: 3, SEQ ID NO: 4, SEQ ID NO: 5, and/or SEQ ID NO: 6.

Also disclosed are methods of any preceding aspect, wherein the cancer is selected from the group consisting of B cell lymphoma, T cell lymphoma, mycosis fungoides, Hodgkin's Disease, myeloid leukemia, squamous cell carcinomas, adenocarcinomas, sarcomas, gliomas, high grade glioma, blastoma, neuroblastomas, osteosarcoma, plasmacytoma, histiocytomas, melanomas, adenomas, hypoxic tumors, myelomas, AIDS-related lymphomas or sarcomas, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, large bowel cancer, hematopoietic cancers; testicular cancer; colon cancer, and rectal cancer.

In one aspect, disclosed herein are methods of modifying an oncolytic virus to inhibit receptor for advanced glycation endproducts (RAGE) interference with the efficacy of the oncolytic virus to clear cancer cells comprising engineering the oncolytic virus to express an endogenous secretory RAGE (esRAGE) gene.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the pathway of HMGB1 activation of the membrane bound RAGE pathway that leads to migration/proliferation, and angiogenesis of the cancer cell and that binding of HMGB1 with esRAGE results in no further signaling and continued oncolytic virus replication.

FIGS. 2A and 2B shows that esRAGE/anti-RAGE treatment reduces oHSV-mediated EC Migration and leakiness. FIG. 2A shows Endothelial cell leakiness was evaluated by measuring the ability of EBA to permeate a confluent endothelial cell (EC) monolayer. Data shown are mean EBA±sd. FIG. 2C shows EC migration after EC were stimulated with CM±oHSV±esRAGE 200 ng/ml or anti-RAGE 2 ug/ml. Data shown are mean number of EC migrated through Transwell membrane±sd.

FIG. 3 shows that OVesRAGE efficiently expresses and secretes esRAGE.

FIG. 4 shows that OVesRAGE significantly inhibits oHSV-induced EC activation and increases oHSV replication.

FIG. 5 shows that OVesRAGE increase glioma cell killing in co-culture with HUVEC.

FIG. 6 shows that OVesRAGE activates NFkB signaling in the macrophage cells.

FIG. 7 shows that OVesRAGE significantly increases macrophage/microglia migration.

FIG. 8 shows the induction and reduction of cytokine expression in U251T3 and BV2 cells following oncolytic virus exposure or modified oncolytic virus expressing esRAGE.

FIG. 9 shows that OVesRAGE significantly increases microglia/macrophage-mediated glioma cell killing.

FIG. 10 shows soluble RAGE (esRAGE) expression results increased survival of infected subjects. DB7 intracranial surgery in Fvbn mice: 2×10\hat{ }5 pfu. OVesRAGE virus IU unit was 20 times higher than control rHSVQ virus, we decide to inject virus with pfu

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular endogenous secretory receptor for advanced glycation endproducts (esRAGE) is disclosed and discussed and a number of modifications that can be made to a number of molecules including the receptor for advanced glycation endproducts (esRAGE) are discussed, specifically contemplated is each and every combination and permutation of receptor for advanced glycation endproducts (esRAGE) and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B—F, C-D, C-E, and C—F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Administration of an oncolytic virus to the forefront of cutting edge cancer therapies. However, the efficacy of oncolytic viral therapy can be inhibited by early innate immune responses to viral infection reduce oHSV replication, tumor destruction, and efficacy. Moreover, inflammatory signals can upregulate expression of the receptor for advanced glycation endproducts (RAGE) on endothelial cells. RAGE is a member of the IgG molecules. The ligands for RAGE include AGE, HMGB1, S100 family, amyloid (3. The interaction between RAGE and its ligands is thought to result in pro-inflammatory gene activation. Binding of any of the RAGE ligands to RAGE causes proliferation, migration, invasion, angiogenesis of endothelial cells. Following oncolytic viral infection, expression of RAGE ligands increases which becomes a significant problem for the efficacy of oncolytic viral treatments providing a mechanism for escape for the cancer cells.

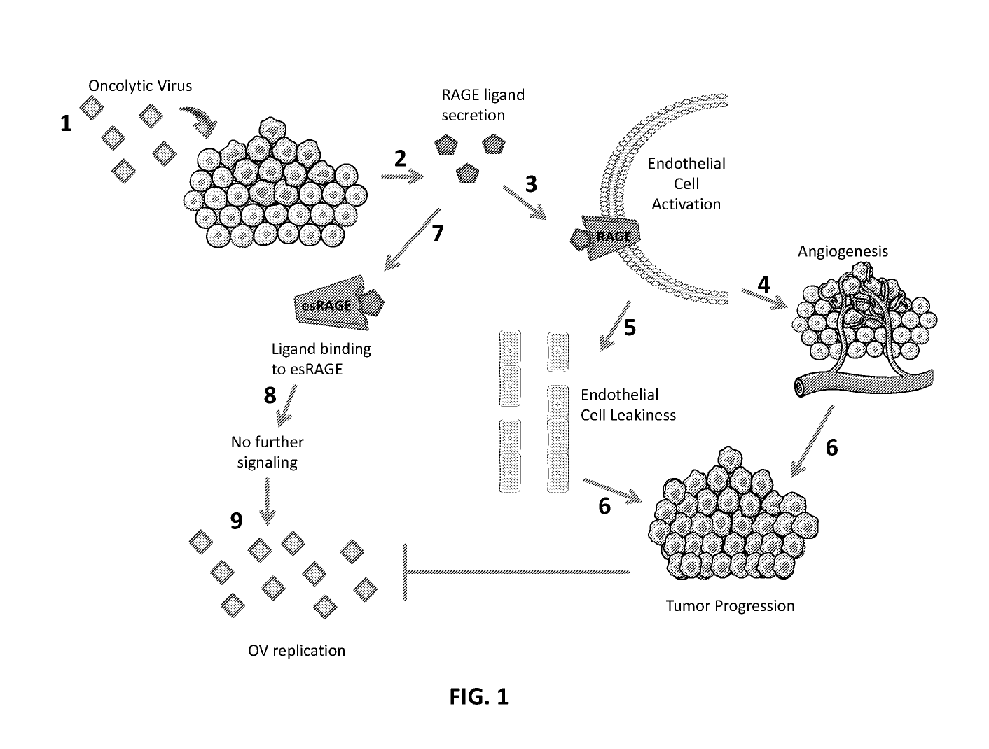

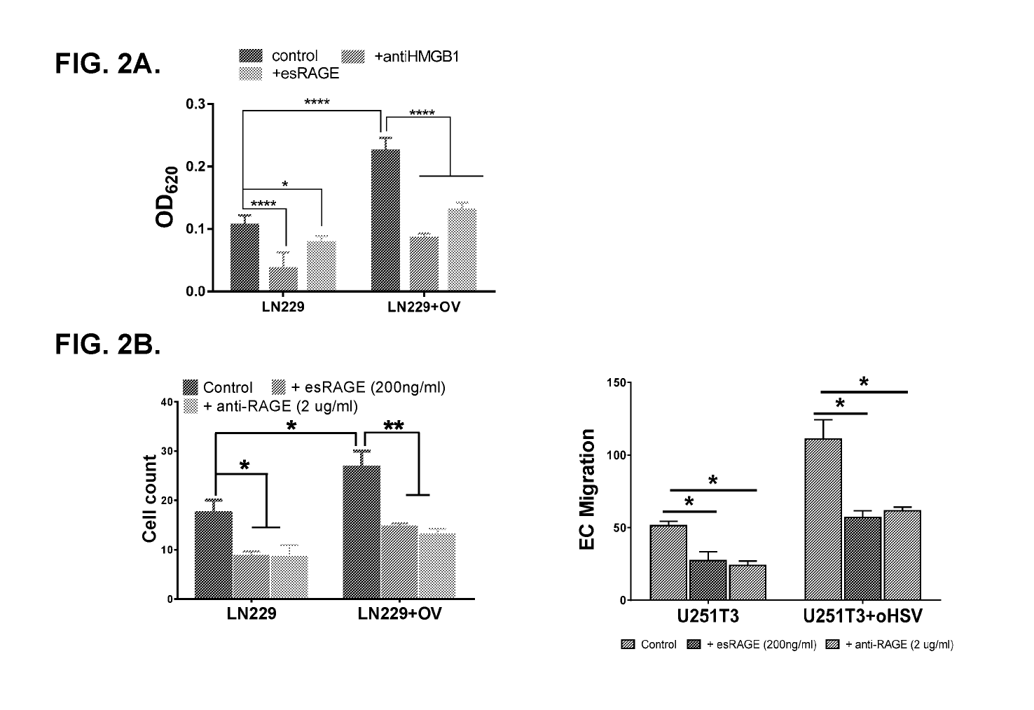

Interestingly, there are several isoforms of the RAGE protein, which lack the transmembrane and the signaling domain (commonly referred to as soluble RAGE or endogenous secretory (esRAGE). These esRAGE peptides, polypeptides, and proteins can also bind RAGE ligands. It is understood and herein contemplated that advantage can be made of the esRAGE proteins to combat the inhibitory effects of membrane bound RAGE. Specifically, esRAGE can compete with membrane bound RAGE for the available ligands which would decrease the amount of ligands able to signal through RAGE. Moreover, as the esRAGe are soluble and thus secreted into the extracellular matrix they would be more bioavailable than membrane bound RAGE and also unable to initiate any cellular signaling cascade (FIG. 1). To test this system, LN229 cells were infected with an oncolytic virus and were either left alone or treated with esRAGE or an anti-RAGE ligand (HGMB1) antibody and measured for endothelial cell (EC) migration and leakiness. Cells receiving either esRAGE of anti-HGMB1 antibody showed decreased leakiness and EC migration (FIG. 2).

In one aspect disclosed herein are modified oncolytic viruses; wherein the oncolytic virus been modified to encode and express the endogenous secretory receptor for advanced glycation endproducts (esRAGE) gene or a functional fragment or variant thereof comprising at least 90% sequence identity to SEQ ID NO: 1, SEQ ID NO: 2, SEQ ID. NO: 3, SEQ ID NO: 4, SEQ ID NO: 5, and/or SEQ ID NO: 6.

In one aspect, the modified oncolytic viruses of any preceding aspect can comprise a viral backbone derived from a modified or engineered Adenovirus, Adeno-associated virus, Herpes Simplex virus-1, Herpes Simplex virus-2, Varicella-Zoster virus, Epstein-Barr virus, Cytomegalovirus, Human Herpes virus-6, Variola virus, Vaccinia virus, Molluscum contagiosum virus, Orf virus, Reovirus, Rotavirus, Enterovirus, Senecavirus, Poliovirus, Coxsackie virus, Rhinovirus, Hepatitis A virus, foot-and-mouth disease virus, Togavirus, Alphavirus, Semliki Forest virus, Eastern Equine Encephalitis virus, Sindbis virus, Rubella virus, Coronavirus, Flavivirus Hepatitis C virus, Japanese Encephalitis virus, St. Louis Encephalitis virus, Murray Valley fever virus, Yellow Fever virus, West Nile virus, Zika virus, Dengue virus, Ebola virus, Marburg virus, Arenavirus, Lassa fever virus, Lymphocytic choriomeningitis virus, Pichinde virus, Junin virus, Machupo virus, Hantaan virus, Rift Valley fever virus, Paramyxovirus, human parainfluenza virus, mumps virus, simian virus 5, measles virus, vesicular stomatitis virus, rabies virus, Respiratory syncytial virus, Orthomyxovirus, Influenza virus A, Influenza virus B, Influenza C virus, Hepatitis D virus, Simian Immunodeficiency virus, Human Immunodeficiency virus type-1, and Human Immunodeficiency virus type-2, Rous sarcoma virus, Human T-cell Leukemia virus type-1 Simian foamy virus, Hepatitis B virus, Hepatitis E virus, Human Papilomavirus, or Polyomavirus. For example, the oncolytic virus can be a Herpes Simplex 1 virus; and wherein the virus is the HSV-1 oncolytic viruses HSV1716, viral ICP34.5 Expressed by Nestin promotor and Vstat120 Expressing; a modified adenovirus oncolytic virus; and wherein the adenovirus is H101; a modified vaccinia virus; and wherein the modified vaccinia viruses is GL-ONC1 or JX-594; a modified reovirus; and wherein the modified reovirus is reolysin; a modified enterovirus, and wherein the modified enterovirus is Riga virus; a modified Senecavirus, and wherein the modified Senecavirus is SVV-001 virus; a modified poliovirus, and wherein the modified poliovirus is PVSRIPO virus; and/or a modified coxsackie virus, and wherein the modified coxsackie virus is A21 virus.

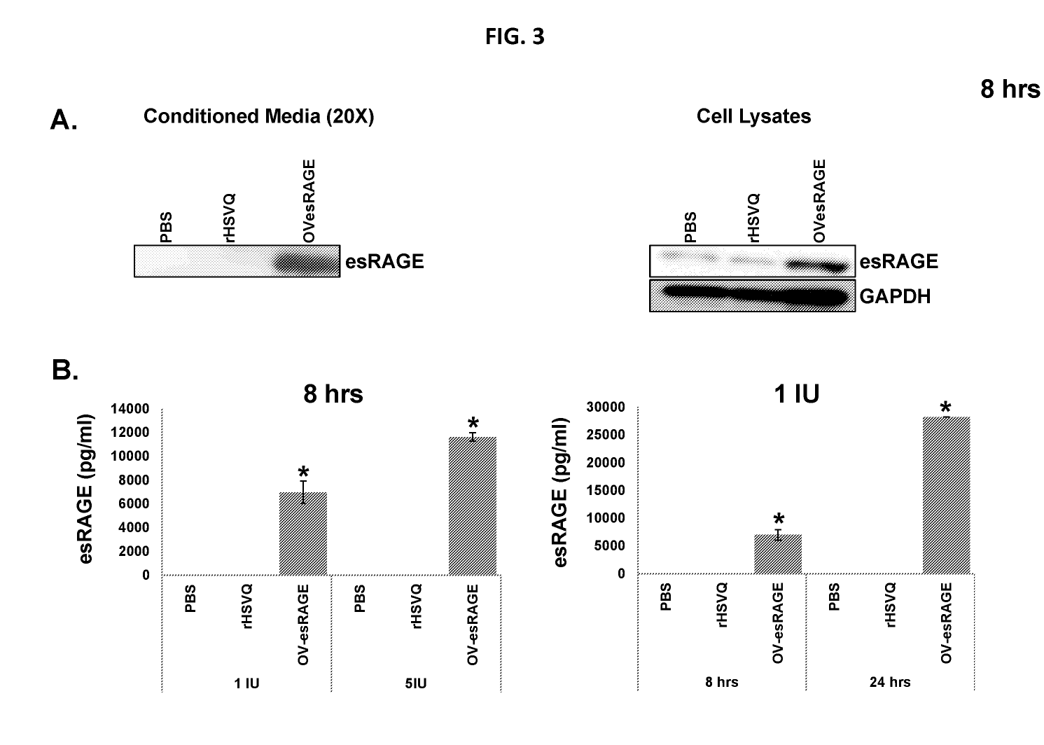

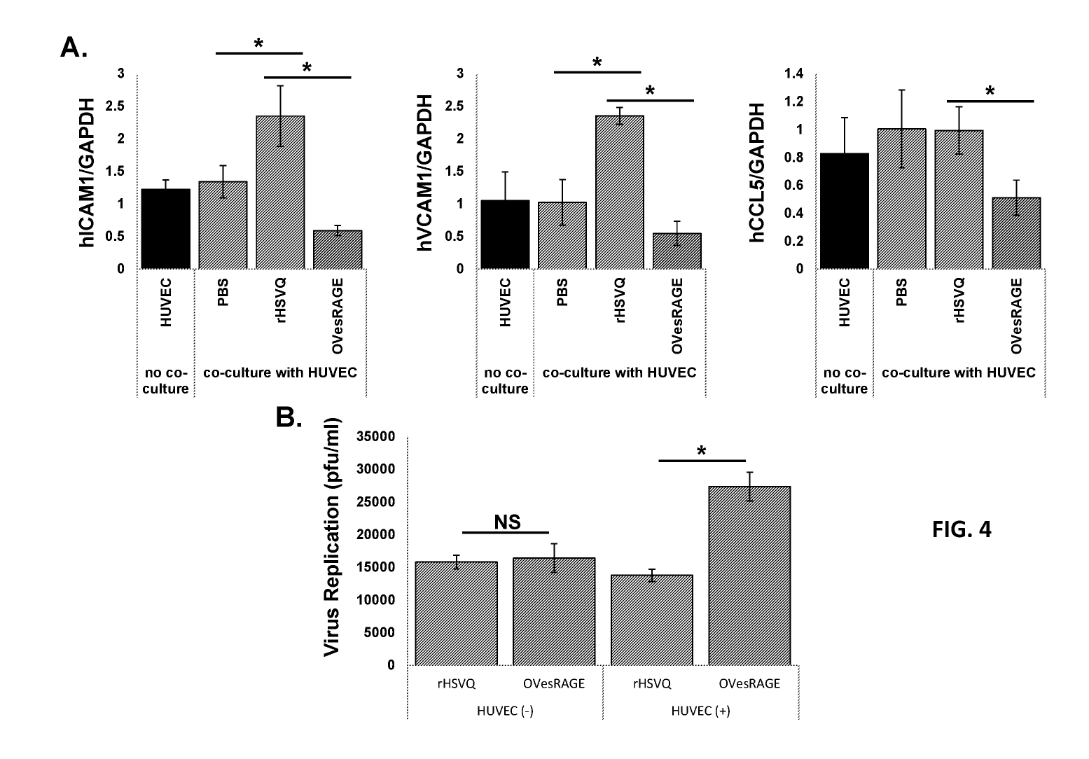

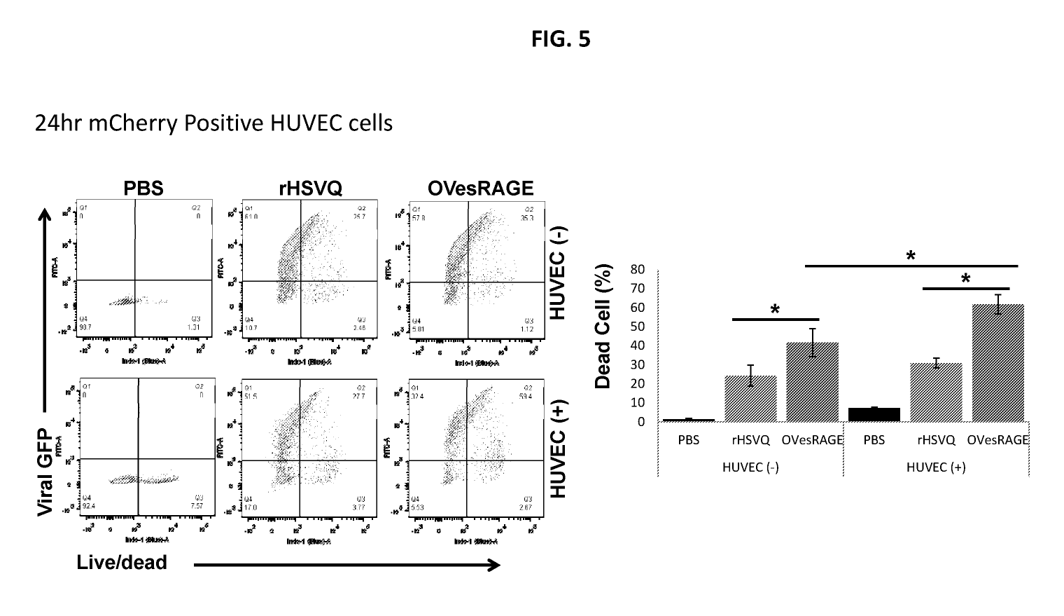

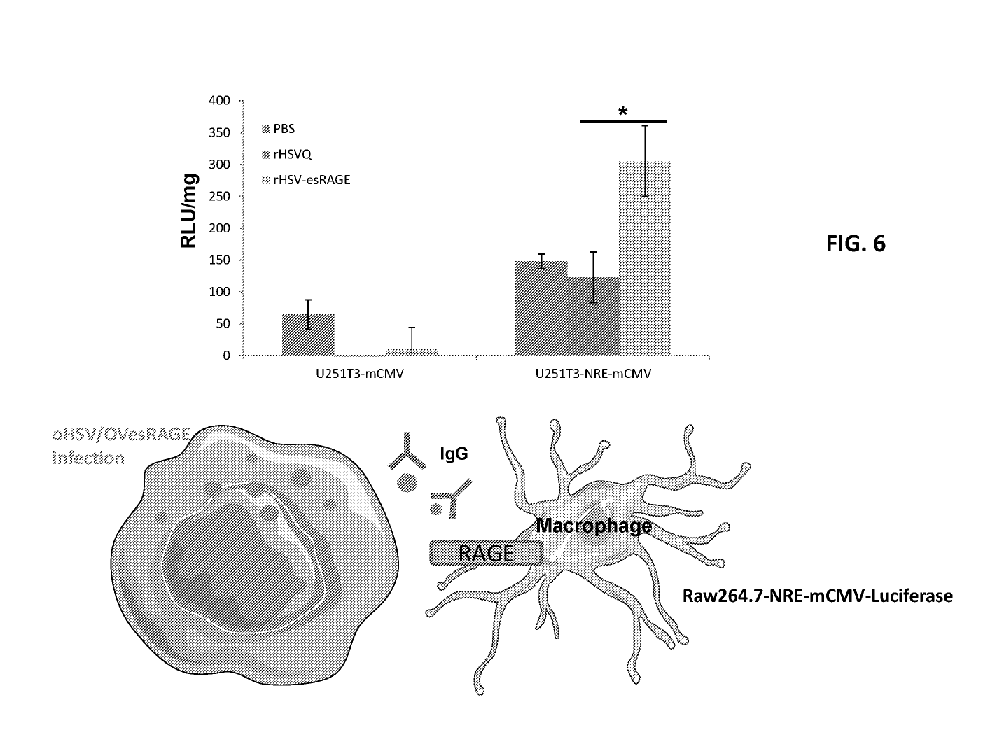

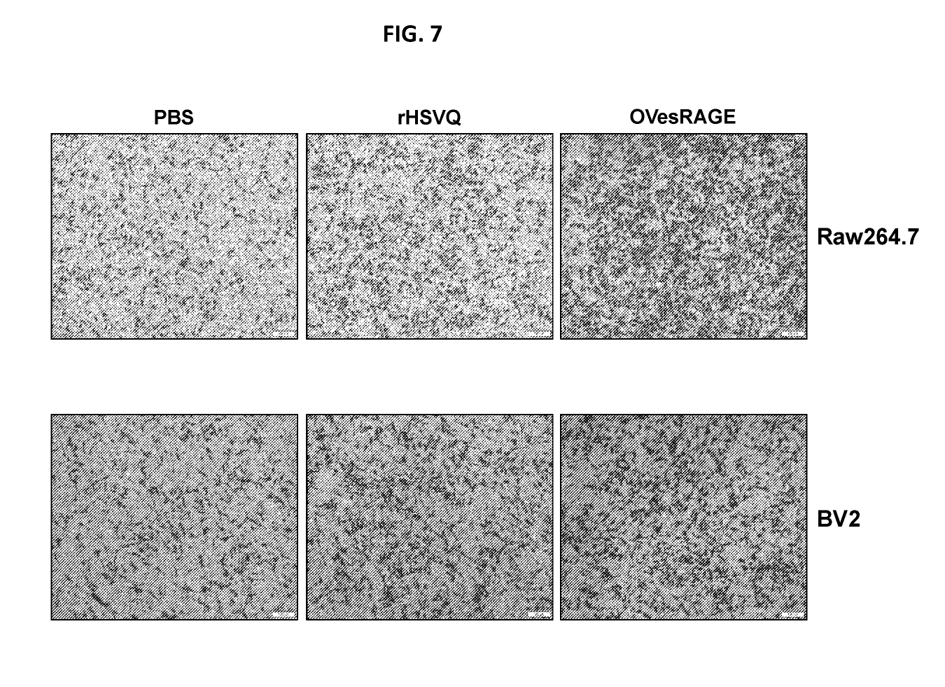

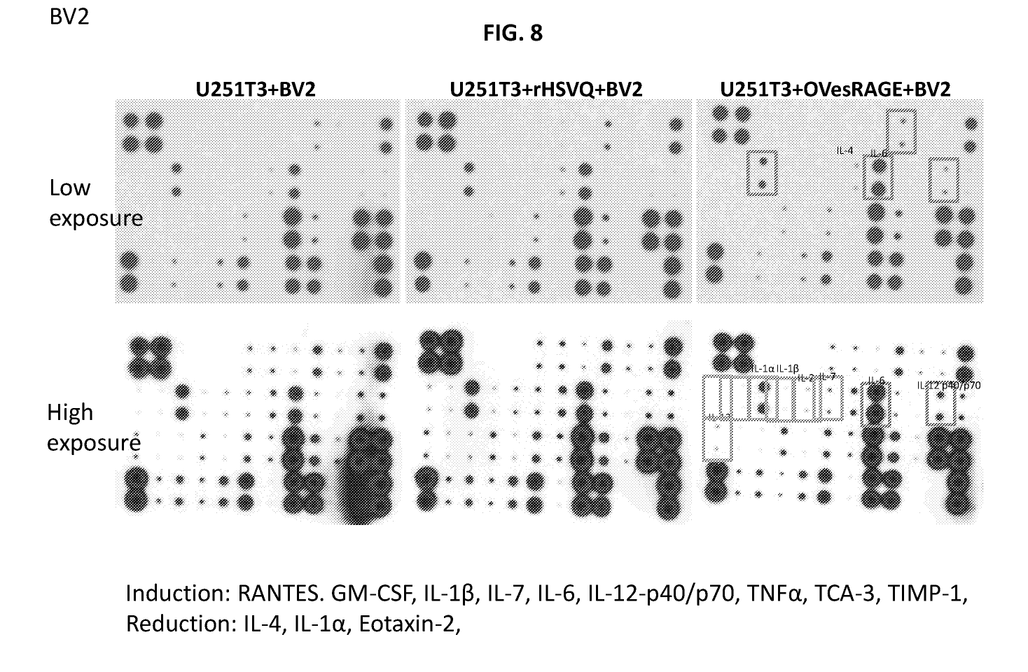

Oncolytic viruses comprising esRAGE were tested for esRAGE expression (FIG. 3). Next the Oncolytic viruses comprising and expressing esRAGE were tested for the ability to inhibit oncolytic virus induced EC activation and for any adverse effects on oncolytic virus replication. The esRAGe expressing oncolytic viruses did not have any effect on viral replication compared to controls, but did reduce oncolytic virus induced EC activation (FIG. 4). The oncolytic viruses comprising esRAGE were then tested for the effect on glioma cell killing. OVesRAGE increased glioma cell killing in co-culture with Human Umbilical Vein Endothelial Cells (HUVEC) (FIG. 5). OVesRAGE also activated NFkB signaling in the macrophage cells (FIG. 6) and significantly increases macrophage/microglia migration (FIG. 7). The viruses were then measured to see their effect on cytokine expression. FIG. 8 shows the induction and reduction of cytokine expression in U251T3 and BV2 cells following oncolytic virus exposure or modified oncolytic virus expressing esRAGE. RANTES. GM-CSF, IL-1β, IL-7, IL-6, IL-12-p40/p70, TNFα, TCA-3, TIMP-1 were all induced while IL-4, IL-la, Eotaxin-2 decreased in expression.

It is understood and herein contemplated that as the disclosed esRAGE oncolytic viruses can successfully thwart the signaling of the RAGE pathway, the method of reduce RAGE interference with oncolytic viral efficacy is valuable. In one aspect, disclosed herein are methods of modifying an oncolytic virus to inhibit receptor for advanced glycation endproducts (RAGE) interference with the efficacy of the oncolytic virus to clear cancer cells comprising engineering the oncolytic virus to express an endogenous secretory RAGE (esRAGE) gene. It is understood and herein contemplated that eh disclosed method of inhibiting RAGE can be performed in any oncolytic virus by modifying the virus to express esRAGE.

1. Homology/Identity

It is understood that one way to define any known variants and derivatives or those that might arise, of the disclosed genes and proteins herein is through defining the variants and derivatives in terms of homology to specific known sequences. For example SEQ ID NOs: 1, 2, 3, 4, 5, and 6 set forth a particular sequence of an esRAGE. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

2. Nucleic Acids

There are a variety of molecules disclosed herein that are nucleic acid based, including for example the nucleic acids that encode, for example esRAGE, or any of the nucleic acids disclosed herein for making oncolytic viruses expressing esRAGE, or fragments thereof, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences related to the protein molecules involved in the signaling pathways disclosed herein, for example esRAGE, or any of the nucleic acids disclosed herein for making esRAGE, all of which are encoded by nucleic acids or are nucleic acids. The sequences for the human analogs of these genes, as well as other analogs, and alleles of these genes, and splice variants and other types of variants, are available in a variety of protein and gene databases, including Genbank. Those of skill in the art understand how to resolve sequence discrepancies and differences and to adjust the compositions and methods relating to a particular sequence to other related sequences.

3. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus or Herpes Simplex Virus-1 is conveniently obtained. Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers f unction to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes ß-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

4. Pharmaceutical Carriers/Delivery of Pharmaceutical Products

As described above, the compositions can also be administered in vivo in a pharmaceutically acceptable carrier. Thus, in one aspect, disclosed herein are pharmaceutical composition comprising a modified oncolytic virus; wherein the oncolytic virus been modified to encode and express the endogenous secretory receptor for advanced glycation endproducts (esRAGE) gene or functional fragment or variant thereof comprising at least 90% sequence identity to SEQ ID NO: 1, SEQ ID NO: 2, SEQ ID. NO: 3, SEQ ID NO: 4, SEQ ID NO: 5, or SEQ ID NO: 6 and a pharmaceutical carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. Methods of Treating Cancer

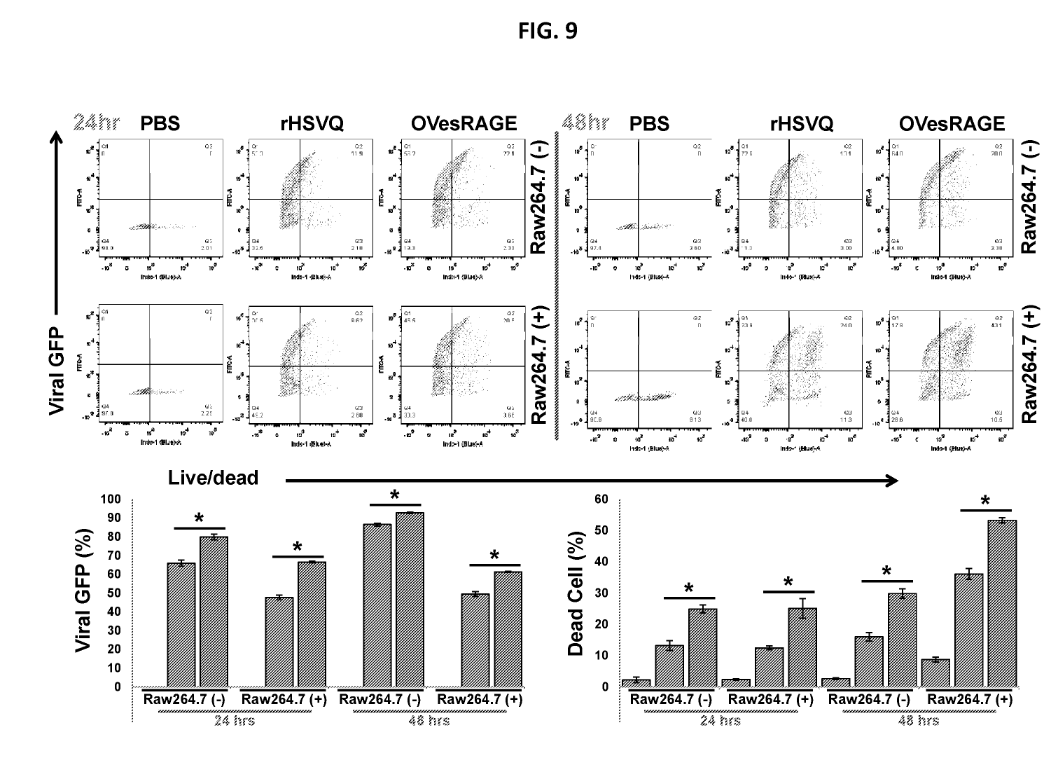

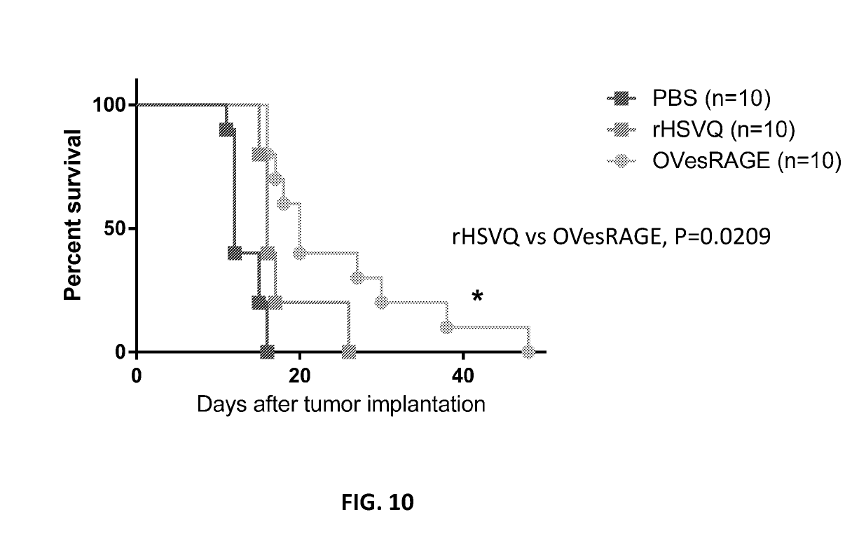

In one aspect, it is understood that the disclosed oncolytic viruses can be used to treat any disease where uncontrolled cellular proliferation occurs such as cancers. As noted herein, the prior art shows that activation inflammatory molecules which bind and signal through membrane bound RAGE leads to proliferation, migration, and angiogenesis of cancer cells. The disclosed oncolytic viruses solve this problem by expressing a soluble (i.e., endogenous secretory) RAGE (esRAGE) which can bind the ligands for RAGE in the extracellular matrix and therefore limit any binding and subsequent signaling through membrane bound RAGE. Expression of esRAGE does not alter NK cell mediated tumor killing. In fact, as shown in FIG. 9 OVesRAGE significantly increases microglia/macrophage-mediated glioma cell killing. This increased tumor targeted killing via soluble RAGE (esRAGE) expression results increased survival of infected subjects (FIG. 10). Accordingly, in one aspect, disclosed herein are methods of treating a subject with cancer comprising administering to the subject any of the esRAGE comprising oncolytic viruses disclosed herein. For example, disclosed herein are methods of treating a subject with a cancer comprising administering to the subject a modified oncolytic virus; wherein the oncolytic virus been modified to encode and express the endogenous secretory receptor for advanced glycation endproducts (esRAGE) gene or a functional fragment thereof comprising at least 90% sequence identity to SEQ ID NO: 1, SEQ ID NO: 2, SEQ ID. NO: 3, SEQ ID NO: 4, SEQ ID NO: 5, or SEQ ID NO: 6.

A non-limiting list of different types of cancers that can be treated using the disclosed oncolytic viruses comprising esRAGE is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, osteosarcoma, plasmacytomas, histiocytomas, melanomas, adenomas, hypoxic tumors, myelomas, AIDS-related lymphomas or sarcomas, metastatic cancers, or cancers in general.

A representative but non-limiting list of cancers that the disclosed compositions can be used to treat is the following: lymphoma, B cell lymphoma, T cell lymphoma, mycosis fungoides, Hodgkin's Disease, myeloid leukemia, squamous cell carcinomas, adenocarcinomas, sarcomas, gliomas, high grade glioma, blastoma, neuroblastomas, osteosarcoma, plasmacytoma, histiocytomas, melanomas, adenomas, hypoxic tumors, myelomas, AIDS-related lymphomas or sarcomas, bladder cancer, brain cancer, nervous system cancer, squamous cell carcinoma of head and neck, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, large bowel cancer, hematopoietic cancers; testicular cancer; colon, and rectal cancers.

It is understood and herein contemplated that the disclosed methods of treating cancers can be applied using any of the oncolytic viruses disclosed herein comprising any oncolytic viral backbone. Accordingly, in one aspect, disclosed herein are methods of treating cancer in a subject comprising administering to the subject a modified oncolytic virus; wherein the oncolytic virus been modified to encode and express the endogenous secretory receptor for advanced glycation endproducts (esRAGE) gene or a functional fragment thereof comprising at least 90% sequence identity to SEQ ID NO: 1, SEQ ID NO: 2, SEQ ID. NO: 3, SEQ ID NO: 4, SEQ ID NO: 5, or SEQ ID NO: 6; wherein the viral backbone of the oncolytic virus is derived from a modified or engineered Adenovirus, Adeno-associated virus, Herpes Simplex virus-1, Herpes Simplex virus-2, Varicella-Zoster virus, Epstein-Barr virus, Cytomegalovirus, Human Herpes virus-6, Variola virus, Vaccinia virus, Molluscum contagiosum virus, Orf virus, Reovirus, Rotavirus, Enterovirus, Senecavirus, Poliovirus, Coxsackie virus, Rhinovirus, Hepatitis A virus, foot-and-mouth disease virus, Togavirus, Alphavirus, Semliki Forest virus, Eastern Equine Encephalitis virus, Sindbis virus, Rubella virus, Coronavirus, Flavivirus Hepatitis C virus, Japanese Encephalitis virus, St. Louis Encephalitis virus, Murray Valley fever virus, Yellow Fever virus, West Nile virus, Zika virus, Dengue virus, Ebola virus, Marburg virus, Arenavirus, Lassa fever virus, Lymphocytic choriomeningitis virus, Pichinde virus, Junin virus, Machupo virus, Hantaan virus, Rift Valley fever virus, Paramyxovirus, human parainfluenza virus, mumps virus, simian virus 5, measles virus, vesicular stomatitis virus, rabies virus, Respiratory syncytial virus, Orthomyxovirus, Influenza virus A, Influenza virus B, Influenza C virus, Hepatitis D virus, Simian Immunodeficiency virus, Human Immunodeficiency virus type-1, and Human Immunodeficiency virus type-2, Rous sarcoma virus, Human T-cell Leukemia virus type-1 Simian foamy virus, Hepatitis B virus, Hepatitis E virus, Human Papilomavirus, or Polyomavirus.

### D. Sequences

| SEQ ID NO: 1 endogenous secretory RAGE variant 1 | |
|---|---|
| 1 | gccaggaccc tggaaggaag caggatggca gccggaacag cagttggagc ctgggtgctg |
| 61 | gtcctcagtc tgtggggggc agtagtaggt gctcaaaaca tcacagcccg gattggcgag |
| 121 | ccactggtgc tgaagtgtaa gggggccccc aagaaaccac cccagcggct ggaatggaaa |
| 181 | ctgaacacag gccggacaga agcttggaag gtcctgtctc cccagggagg aggcccctgg |
| 241 | gacagtgtgg ctcgtgtcct tcccaacggc tccctcttcc ttccggctgt cgggatccag |
| 301 | gatgagggga ttttccggtg ccaggcaatg aacaggaatg gaaaggagac caagtccaac |
| 361 | taccgagtcc gtgtctacca gattcctggg aagccagaaa ttgtagattc tgcctctgaa |
| 421 | ctcacggctg gtgttcccaa taaggtgggg acatgtgtgt cagagggaag ctaccctgca |
| 481 | gggactctta gctggcactt ggatgggaag cccctggtgc ctaatgagaa gggagtatct |
| 541 | gtgaaggaac agaccaggag acaccctgag acagggctct tcacactgca gtcggagcta |
| 601 | atggtgaccc cagcccgggg aggagatccc cgtcccacct tctcctgtag cttcagccca |
| 661 | ggccttcccc gacaccgggc cttgcgcaca gcccccatcc agccccgtgt ctgggagcct |
| 721 | gtgcctctgg aggaggtcca attggtggtg gagccagaag gtggagcagt agctcctggt |
| 781 | ggaaccgtaa ccctgacctg tgaagtccct gcccagccct ctcctcaaat ccactggatg |
| 841 | aaggatggtg tgcccttgcc ccttcccccc agccctgtgc tgatcctccc tgagataggg |
| 901 | cctcaggacc agggaaccta cagctgtgtg gccacccatt ccagccacgg gccccaggaa |
| 961 | agccgtgctg tcagcatcag catcatcgaa ccaggcgagg aggggccaac tgcaggtgag |
| 1021 | gggtttgata aagtcaggga agcagaagat agcccccaac acatgtgact ggggggatgg |
| 1081 | tcaacaagaa aggaatggaa ggccccagaa aaccaggagg aagaggagga gcgtgcagaa |
| 1141 | ctgaatcagt cggaggaacc tgaggcaggc gagagtagta ctggagggcc ttgaggggcc |
| 1201 | cacagacaga tcccatccat cag |
| SEQ ID NO: 2: endogenous secretory RAGE variant 2 | |
| 1 | aggaagcagg atggcagccg gaacagcagt tggagcctgg gtgctggtcc tcagtctgtg |
| 61 | gggggcagta gtaggtgctc aaaacatcac agcccggatt ggcgagccac tggtgctgaa |
| 121 | gtgtaagggg gcccccaaga aaccacccca gcggctggaa tggaaactga acacaggccg |
| 181 | gacagaagct tggaaggtcc tgtctcccca gggaggaggc ccctgggaca gtgtggctcg |
| 241 | tgtccttccc aacggctccc tcttccttcc ggctgtcggg atccaggatg aggggatttt |
| 301 | ccggtgccag gcaatgaaca ggaatggaaa ggagaccaag tccaactacc gagtccgtgt |
| 361 | ctaccagatt cctgggaagc cagaaattgt agattctgcc tctgaactca cggctggtgt |
| 421 | tcccaataag gtggggacat gtgtgtcaga gggaagctac cctgcaggga ctcttagctg |
| 481 | gcacttggat gggaagcccc tggtgcctaa tgagaaggga gtatctgtga aggaacagac |
| 541 | caggagacac cctgagacag ggctcttcac actgcagtcg gagctaatgg tgaccccagc |
| 601 | ccggggagga gatccccgtc ccaccttctc ctgtagcttc agcccaggcc ttccccgaca |
| 661 | ccgggccttg cgcacagccc ccatccagcc ccgtgtctgg gagcctgtgc ctctggagga |
| 721 | ggtccaattg gtggtggagc cagaaggtgg agcagtagct cctggtggaa ccgtaaccct |
| 781 | gacctgtgaa gtccctgccc agccctctcc tcaaatccac tggatgaagg atggcctcag |
| 841 | gaccagggaa cctacagctg tgtggccacc cattccagcc acgggcccca ggaaagccgt |
| 901 | gctgtcagca tcagcatcat cgaaccaggc gaggaggggc caactgcagg ctctgtggga |
| 961 | ggatcagggc tgggaactct agccctggcc ctggggatcc tgggaggcct ggggacagcc |
| 1021 | gccctgctca ttggggtcat cttgtggcaa aggcggcaac gccgaggaga ggagaggaag |
| 1081 | gccccagaaa accaggagga agaggaggag cgtgcagaac tgaatcagtc ggaggaacct |
| 1141 | gaggcaggcg agagtagtac tggagggcct tgaggggccc acagacagat ccca |
| SEQ ID NO: 3: endogenous secretory RAGE variant 3 | |
| 1 | aggaagcagg atggcagccg gaacagcagt tggagcctgg gtgctggtcc tcagtctgtg |
| 61 | gggggcagta gtaggtgctc aaaacatcac agcccggatt ggcgagccac tggtgctgaa |
| 121 | gtgtaagggg gcccccaaga aaccacccca gcggctggaa tggaaactga acacaggccg |
| 181 | gacagaagct tggaaggtcc tgtctcccca gggaggaggc ccctgggaca gtgtggctcg |
| 241 | tgtccttccc aacggctccc tcttccttcc ggctgtcggg atccaggatg aggggatttt |
| 301 | ccggtgccag gcaatgaaca ggaatggaaa ggagaccaag tccaactacc gagtccgtgt |
| 361 | ctaccagatt cctgggaagc cagaaattgt agattctgcc tctgaactca cggctggtgt |
| 421 | tcccaataag gtggggacat gtgtgtcaga gggaagctac cctgcaggga ctcttagctg |
| 481 | gcacttggat gggaagcccc tggtgcctaa tgagaaggga gtatctgtga aggaacagac |
| 541 | caggagacac cctgagacag ggctcttcac actgcagtcg gagctaatgg tgaccccagc |
| 601 | ccggggagga gatccccgtc ccaccttctc ctgtagcttc agcccaggcc ttccccgaca |
| 661 | ccgggccttg cgcacagccc ccatccagcc ccgtgtctgg gagcctgtgc ctctggagga |
| 721 | ggtccaattg gtggtggagc cagaaggtgg agcagtagct cctggtggaa ccgtaaccct |
| 781 | gacctgtgaa gtccctgccc agccctctcc tcaaatccac tggatgaagg atggtgtgcc |
| 841 | cttgcccctt ccccccagcc ctgtgctgat cctccctgag atagggcctc aggaccaggg |
| 901 | aacctacagc tgtgtggcca cccattccag ccacgggccc caggaaagcc gtgctgtcag |
| 961 | catcagcatc atcgaaccag gcgaggaggg gccaactgca ggtgaggggt ttgataaagt |
| 1021 | cagggaagca gaagatagcc cccaacacat gtgactgggg ggatggtcaa caagaaagga |
| 1081 | atggaaggcc ccagaaaacc aggaggaaga ggaggagcgt gcagaactga atcagtcgga |
| 1141 | ggaacctgag gcaggcgaga gtagtactgg agggccttga ggggcccaca gacagatccc |
| 1201 | a |
| SEQ ID NO: 4: endogenous secretory RAGE variant 4 | |
| 1 | aggaagcagg atggcagccg gaacagcagt tggagcctgg gtgctggtcc tcagtctgtg |
| 61 | gggggcagta gtaggtgctc aaaacatcac agcccggatt ggcgagccac tggtgctgaa |
| 121 | gtgtaagggg gcccccaaga aaccacccca gcggctggaa tggaaactga acacaggccg |
| 181 | gacagaagct tggaaggtcc tgtctcccca gggaggaggc ccctgggaca gtgtggctcg |
| 241 | tgtccttccc aacggctccc tcttccttcc ggctgtcggg atccaggatg aggggatttt |
| 301 | ccggtgccag gcaatgaaca ggaatggaaa ggagaccaag tccaactacc gagtccgtgt |
| 361 | ctaccagatt cctgggaagc cagaaattgt agattctgcc tctgaactca cggctggtgt |
| 421 | tcccaataag gtggggacat gtgtgtcaga gggaagctac cctgcaggga ctcttagctg |
| 481 | gcacttggat gggaagcccc tggtgcctaa tgagaaggga gtatctgtga aggaacagac |
| 541 | caggagacac cctgagacag ggctcttcac actgcagtcg gagctaatgg tgaccccagc |
| 601 | ccggggagga gatccccgtc ccaccttctc ctgtagcttc agcccaggcc ttccccgaca |
| 661 | ccgggccttg cgcacagccc ccatccagcc ccgtgtctgg gagcctgtgc ctctggagga |
| 721 | ggtccaattg gtggtggagc cagaaggtgg agcagtagct cctggtggaa ccgtaaccct |
| 781 | gacctgtgaa gtccctgccc agccctctcc tcaaatccac tggatgaagg atggcctcag |
| 841 | gaccagggaa cctacagctg tgtggccacc cattccagcc acgggcccca ggaaagccgt |
| 901 | gctgtcagca tcagcatcat cgaaccaggc gaggaggggc caactgcagg tgaggggttt |
| 961 | gataaagtca gggaagcaga agatagcccc caacacatgt gactgggggg atggtcaaca |
| 1021 | agaaaggaat ggaaggcccc agaaaaccag gaggaagagg aggagcgtgc agaactgaat |
| 1081 | cagtcggagg aacctgaggc aggcgagagt agtactggag ggccttgagg ggcccacaga |
| 1141 | cagatccca |
| SEQ ID NO: 5: endogenous secretory RAGE variant 5 | |
| 1 | aggaagcagg atggcagccg gaacagcagt tggagcctgg gtgctggtcc tcagtctgtg |
| 61 | gggggcagta gtaggtgctc aaaacatcac agcccggatt ggcgagccac tggtgctgaa |
| 121 | gtgtaagggg gcccccaaga aaccacccca gcggctggaa tggaaactga acacaggccg |
| 181 | gacagaagct tggaaggtcc tgtctcccca gggaggaggc ccctgggaca gtgtggctcg |
| 241 | tgtccttccc aacggctccc tcttccttcc ggctgtcggg atccaggatg aggggatttt |
| 301 | ccggtgccag gcaatgaaca ggaatggaaa ggagaccaag tccaactacc gagtccgtgt |
| 361 | ctaccagatt cctgggaagc cagaaattgt agattctgcc tctgaactca cggctggtgt |
| 421 | tcccaataag gtagtggaag aaagcaggag aagtagaaaa cggccctgtg aacaggaggt |
| 481 | ggggacatgt gtgtcagagg gaagctaccc tgcagggact cttagctggc acttggatgg |
| 541 | gaagcccctg gtgcctaatg agaagggagt atctgtgaag gaacagacca ggagacaccc |
| 601 | tgagacaggg ctcttcacac tgcagtcgga gctaatggtg accccagccc ggggaggaga |
| 661 | tccccgtccc accttctcct gtagcttcag cccaggcctt ccccgacacc gggccttgcg |
| 721 | cacagccccc atccagcccc gtgtctggga gcctgtgcct ctggaggagg tccaattggt |
| 781 | ggtggagcca gaaggtggag cagtagctcc tggtggaacc gtaaccctga cctgtgaagt |
| 841 | ccctgcccag ccctctcctc aaatccactg gatgaaggat ggtgtgccct tgccccttcc |
| 901 | ccccagccct gtgctgatcc tccctgagat agggcctcag gaccagggaa cctacagctg |
| 961 | tgtggccacc cattccagcc acgggcccca ggaaagccgt gctgtcagca tcagcatcat |
| 1021 | cgaaccaggc gaggaggggc caactgcagg tgaggggttt gataaagtca gggaagcaga |
| 1081 | agatagcccc caacacatgt gactgggggg atggtcaaca agaaaggaat ggaaggcccc |
| 1141 | agaaaaccag gaggaagagg aggagcgtgc agaactgaat cagtcggagg aacctgaggc |
| 1201 | aggcgagagt agtactggag ggccttgagg ggcccacaga cagatccca |
| SEQ ID NO: 6: endogenous secretory RAGE variant 6 | |
| 1 | atggcagccg gaacagcagt tggagcctgg gtgctggtcc tcagtctgtg gggggcagta |
| 61 | gtaggtgctc aaaacatcac agcccggatt ggcgagccac tggtgctgaa gtgtaagggg |
| 121 | gcccccaaga aaccacccca gcggctggaa tggaaactga acacaggccg gacagaagct |
| 181 | tggaaggtcc tgtctcccca gggaggaggc ccctgggaca gtgtggctcg tgtccttccc |
| 241 | aacggctccc tcttccttcc ggctgtcggg atccaggatg aggggatttt ccggtgccag |
| 301 | gcaatgaaca ggaatggaaa ggagaccaag tccaactacc gagtccgtgt ctaccagatt |
| 361 | cctgggaagc cagaaattgt agattctgcc tctgaactca cggctggtgt tcccaataag |
| 421 | gtggggacat gtgtgtcgga gggaagctac cctgcaggga ctcttagctg gcacttggat |
| 481 | gggaagcccc tggtgcctaa tgagaaggga gtatctgtga aggaacagac caggagacac |
| 541 | cctgagacag ggctcttcac actgcagtcg gagctaatgg tgaccccagc ccggggagga |
| 601 | gatccccgtc ccaccttctc ctgtagcttc agcccaggcc ttccccgaca ccgggccttg |
| 661 | cgcacagccc ccatccagcc ccgtgtctgg gagcctgtgc ctctggagga ggtccaattg |
| 721 | gtggtggagc cagaaggtgg agcagtagct cctggtggaa ccgtaaccct gacctgtgaa |
| 781 | gtccctgccc agccctctcc tcaaatccac tggatgaagg atggggtgcc cttgcccctt |
| 841 | ccccccagcc ctgtgctgat cctccctgag atagggcctc aggaccaggg aacctacagc |
| 901 | tgtgtggcca cccattccag ccacgggccc caggaaagcc gtgctgtcag catcagcatc |
| 961 | atcggtgaga cctctcccca agccctacag accctgggac tagggtgcag gacagcacag |
| 1021 | gctctaattt cctgccccat tctggcctta tccctaacag ccaccccacc tctccctcca |
| 1081 | tgcacccaca cccaagcctc ccctgcccca cccaaattct gccaagagag cagccaagcc |
| 1141 | tctcccttct tccctctgag ctaa |

1. A modified oncolytic virus; wherein the oncolytic virus been modified to encode and express the endogenous secretory receptor for advanced glycation endproducts (esRAGE) gene or functional fragment or variant thereof comprising at least 90% sequence identity to SEQ ID NO: 1, SEQ ID NO: 2, SEQ ID. NO: 3, SEQ ID NO: 4, SEQ ID NO: 5, or SEQ ID NO: 6.

2. The modified oncolytic virus of claim 1, wherein the viral backbone is derived from a modified or engineered Adenovirus, Adeno-associated virus, Herpes Simplex virus-1, Herpes Simplex virus-2, Varicella-Zoster virus, Epstein-Barr virus, Cytomegalovirus, Human Herpes virus-6, Variola virus, Vaccinia virus, Molluscum contagiosum virus, Orf virus, Reovirus, Rotavirus, Enterovirus, Senecavirus, Poliovirus, Coxsackie virus, Rhinovirus, Hepatitis A virus, foot-and-mouth disease virus, Togavirus, Alphavirus, Semliki Forest virus, Eastern Equine Encephalitis virus, Sindbis virus, Rubella virus, Coronavirus, Flavivirus Hepatitis C virus, Japanese Encephalitis virus, St. Louis Encephalitis virus, Murray Valley fever virus, Yellow Fever virus, West Nile virus, Zika virus, Dengue virus, Ebola virus, Marburg virus, Arenavirus, Lassa fever virus, Lymphocytic choriomeningitis virus, Pichinde virus, Junin virus, Machupo virus, Hantaan virus, Rift Valley fever virus, Paramyxovirus, human parainfluenza virus, mumps virus, simian virus 5, measles virus, vesicular stomatitis virus, rabies virus, Respiratory syncytial virus, Orthomyxovirus, Influenza virus A, Influenza virus B, Influenza C virus, Hepatitis D virus, Simian Immunodeficiency virus, Human Immunodeficiency virus type-1, and Human Immunodeficiency virus type-2, Rous sarcoma virus, Human T-cell Leukemia virus type-1 Simian foamy virus, Hepatitis B virus, Hepatitis E virus, Human Papilomavirus, or Polyomavirus.

3. The modified oncolytic virus of claim 2, wherein the oncolytic virus is a Herpes Simplex 1 virus; and wherein the virus is the HSV-1 oncolytic viruses HSV1716, viral ICP34.5 Expressed by Nestin promotor and Vstat120 Expressing.

4. The modified oncolytic virus of claim 2, wherein the oncolytic virus is a modified adenovirus oncolytic virus; and wherein the adenovirus is H101.

5. The modified oncolytic virus of claim 2, wherein the oncolytic virus is a modified vaccinia virus; and wherein the modified vaccinia viruses is GL-ONC1 or JX-594.

6. The modified oncolytic virus of claim 2, wherein the oncolytic virus is a modified reovirus; and wherein the modified reovirus is reolysin.

7. The modified oncolytic virus of claim 2, wherein the oncolytic virus is a modified enterovirus, and wherein the modified enterovirus is Riga virus.

8. The modified oncolytic virus of claim 2, wherein the oncolytic virus is a modified Senecavirus, and wherein the modified Senecavirus is SVV-001 virus.

9. The modified oncolytic virus of claim 2, wherein the oncolytic virus is a modified poliovirus, and wherein the modified poliovirus is PVSRIPO virus.

10. The modified oncolytic virus of claim 2, wherein the oncolytic virus is a modified coxsackie virus, and wherein the modified coxsackie virus is A21 virus.

11. A pharmaceutical composition comprising the oncolytic virus of claim 1 and a pharmaceutical carrier.

12. A method of treating a subject with cancer comprising administering to the subject the oncolytic virus of claim 1.

13. A method of treating a subject with a cancer comprising administering to the subject modified oncolytic virus; wherein the oncolytic virus been modified to encode and express the endogenous secretory receptor for advanced glycation endproducts (esRAGE) gene or a functional fragment or variant thereof comprising at least 90% sequence identity to SEQ ID NO: 1, SEQ ID NO: 2, SEQ ID. NO: 3, SEQ ID NO: 4, SEQ ID NO: 5, or SEQ ID NO: 6.

14. The method of claim 13, wherein the cancer is selected from the group consisting of B cell lymphoma, T cell lymphoma, mycosis fungoides, Hodgkin's Disease, myeloid leukemia, squamous cell carcinomas, adenocarcinomas, sarcomas, gliomas, high grade glioma, blastoma, neuroblastomas, osteosarcoma, plasmacytoma, histiocytomas, melanomas, adenomas, hypoxic tumors, myelomas, AIDS-related lymphomas or sarcomas, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, large bowel cancer, hematopoietic cancers; testicular cancer; colon cancer, and rectal cancer.

15. A method of modifying an oncolytic virus to inhibit receptor for advanced glycation endproducts (RAGE) interference with the efficacy of the oncolytic virus to clear cancer cells comprising engineering the oncolytic virus to express an endogenous secretory RAGE (esRAGE) gene.
